(19) United States
(12) Patent Application Publication
(10) Pub. No.: US 2016/0106331 A1
(43) Pub. Date: Apr. 21, 2016

(54) FRACTAL INDEX ANALYSIS OF HUMAN ELECTROENCEPHALOGRAM SIGNALS

(71) Applicants: THE REGENTS OF THE UNIVERSITY OF CALIFORNIA, Oakland, CA (US); THE U.S. GOVERNMENT REPRESENTED BY THE DEPARTMENT OF VETERANS AFFAIRS, Washington, DC (US)

(72) Inventors: Todd S. Zorick, Long Beach, CA (US); Mark Mandelkern, Pacific Palisades, CA (US)

(73) Assignees: THE REGENTS OF THE UNIVERSITY OF CALIFORNIA, Oakland, CA (US); THE U.S. GOVERNMENT REPRESENTED BY THE DEPARTMENT OF VETERANS AFFAIRS, Washington, DC (US)

(21) Appl. No.: 14/919,702

(22) Filed: Oct. 21, 2015

Related U.S. Application Data
(63) Continuation of application No. PCT/US2014/035045, filed on Apr. 22, 2014.
(60) Provisional application No. 61/814,382, filed on Apr. 22, 2013.

(51) Int. Cl. A61B 5/04; A61B 5/16; A61B 5/00; A61B 5/0476

(52) U.S. Cl. CPC A61B 5/04012; A61B 5/0476; A61B 5/16; A61B 5/4806; A61B 5/7253

(57) ABSTRACT

A system and method for Multifractal-Detrended Fluctuation Analysis (MF-DFA) on digitized Human EEG signals is presented. A list of Hurst exponents, or Hurst exponent spectrum (“h” values) are generated, and multifractal singularity spectrum indices (“D(h)” values) produce a graph that approximates an inverted parabola. The output multifractal DFA spectrum is able to represent key features of the internal neuronal dynamics for the cortical neurons underlying the scalp-placed electrode which records the signals.

### CROSS-REFERENCE TO RELATED APPLICATIONS

This application is a 35 U.S.C. §111(a) continuation of PCT international application number PCT/US2014/035045 filed on Apr. 22, 2014, incorporated herein by reference in its entirety, which claims priority to, and the benefit of, U.S. provisional patent application Ser. No. 61/814,382 filed on Apr. 22, 2013, incorporated herein by reference in its entirety. Priority is claimed to each of the foregoing applications.

The above-referenced PCT international application was published as PCT International Publication No. WO 2014/176286 on Oct. 30, 2014, which publication is incorporated herein by reference in its entirety.

### STATEMENT REGARDING FEDERALLY SPONSORED RESEARCH OR DEVELOPMENT

This work was supported by the U.S. Department of Veterans Affairs. The Government has certain rights in the invention.

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to analysis of electroencephalography (EEG) signals, and more particularly to fractal index analysis of EEG signals.

2. Description of Related Art

While human electroencephalography (EEG) recordings have been utilized for clinical and research purposes since the 1920s, still much is unknown about the underlying neuronal dynamics responsible for scalp-recorded electric potential changes as a function of time. Based upon the physiological and conductive properties of the intervening scalp and skull, EEG electrodes are thought to record space-averaged electrical potentials representing synaptic activity of 108-109 cortical neurons, therefore with poor spatial resolution, but excellent temporal resolution compared to other neuroimaging modalities. Current clinical uses of EEG involve spectral analysis via Fourier transform, which can accurately decompose underlying signal frequencies of a stationary signal.

Current state-of-the-art for clinical EEG analysis involves time-averaged spectral analysis, i.e. looking for the strongest frequency band in either delta (<4 Hz), theta (4-7 Hz), alpha (8-13 Hz), beta-gamma (>14 Hz), specifically without any mention of nonlinear methods to analyze EEG signals.

### BRIEF SUMMARY OF THE INVENTION

An aspect of the present invention is a system and method for Multifractal-Detrended Fluctuation Analysis (MF-DFA) on digitized Human EEG signals. Using the system and method of the present invention on lengths of EEG signals (e.g. >5 seconds collected at 256 Hz), a list of Hurst exponents (“Hurst exponent spectrum” or “h” values) are generated, and multifractal singularity spectrum indices (“D(h)” values) produce a graph that approximates an inverted parabola. This “multifractal DFA spectrum” of h vs. D(h) values is able to represent key features of the internal neuronal dynamics for the cortical neurons underlying the scalp-placed electrode which records the signals. For instance, in waking EEG states, both within-subject and between-subject variances for the parameters that characterize the MF-DFA spectrum are very low, indicating the effectiveness of the present method at characterizing intrinsic neuronal cortical dynamics.

An aspect of the present invention is a system and method to identify and distinguish patterns of cortical neuronal dynamics among patients with neurological disorders and psychiatric disorders. The system and method of the present invention may include embodiments having specific applicability in the automatic distinguishing of seizure states, sleep stages, states of anesthesia, neurological illness, or psychiatric illness.

The system and method of the present invention may be employed in clinical neuroscience for treatment settings in psychiatry, psychology, and neurology, etc. If used in psychiatry, for instance, the system and method of the present invention may be implemented for virtually every patient referred to psychiatry and/or psychology to have a diagnostic EEG performed on them, and their resulting multifractal DFA spectrum information would provide valuable assistance in diagnosis and treatment of the patients. Subsequent diagnostic multifractal DFA spectrum testing in accordance with the present invention may then quantify treatment results, or assess for change in clinical status at a subsequent time.

The system and method of the present invention may also be employed for application automatic detection of sleep stages in sleep medicine, status of artificially induced coma in anesthesia, and seizure states in neurology, or other areas of clinical neuroscience.

Another aspect is an EEG reader configured to acquire EEG signals from a patient, and report back a classification of the subject's underlying neuronal dynamics, based upon analysis of the patient's multifractal DFA spectrum and comparison with a known database of multifractal DFA spectrum information from a collection of patients with (and without) known neurological and psychiatric disease.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 shows a schematic diagram of a system configured to record and analyze an individual's EEG according to the methods of the present invention.

FIG. 2 is a flow diagram of a method for reading an individual's EEG based upon the multifractal DFA spectra.

FIG. 3 shows a plot of the average multifractal DFA spectrum obtained from 13 subjects, 1 minute of waking EEG per subject, showing the inverse parabola distribution of data for h versus D(h) plots.

FIG. 4 shows a plot of the average multifractal DFA spectrum obtained from 12 different 30 second segments of waking EEG from a single subject.

FIG. 5 is a plot of an exemplary multifractal DFA spectrum from a subject with waking EEG versus a subject having a witnessed seizure.

FIG. 6 is a plot of multifractal DFA spectra in different stages of sleep.

FIG. 7 shows a diagram of an exemplary classification tree algorithm for distinguishing sleep stages from multifractal DFA spectra of human EEG in accordance with the present invention.

FIG. 8 is a plot showing a comparison of MF-DFA spectrum from waking EEG to numerical models of mono- and multifractal processes.

FIG. 9A and FIG. 9B show plots of a variance comparison between MF-DFA and WTMM techniques for 14 subjects with 8 m of waking EEG.

FIG. 10 is a plot comparing MF-DFA spectra of waking and sleep stage 2.

FIG. 11 illustrates a plot of MF-DFA spectra used in evaluation of schizophrenia.

FIG. 12 illustrates a plot of MF-DFA spectra used in evaluation of delirium.

FIG. 13 illustrates a plot of MF-DFA spectra used in evaluation of Traumatic Brain Injury (TBI).

FIG. 14 illustrates a plot of MF-DFA spectra used in evaluation of Dementia and Mild Cognitive Impairment (MCI).

### DETAILED DESCRIPTION OF THE INVENTION

A basic premise of the present invention is that the underlying pattern of neuronal activation which results in EEG trace recordings is nonlinear, with scale-free dynamics, while EEG signals themselves are nonstationary. Therefore, traditional statistical methods of EEG analysis (e.g., Fourier Transform, spectral analysis) may not be the most relevant means to analyze EEG signals, since these techniques would miss many properties inherent in nonstationary signals with scale-free (self-affine) dynamics.

According to methods and systems of the present invention, MF-DFA of scalp EEG signals recorded from humans are used to gain an improved understanding of the relevant underlying neuronal dynamics. Given that cortical neuronal networks exhibit nonlinear interactions characterized by a range of fractal exponents with varying scales, the MF-DFA techniques of the present invention are capable of describing essential features of the underlying neuronal dynamics for EEG signals in a way that is superior either to traditional techniques (e.g., spectral analysis via fourier transform), or measures derived from monofractal analysis (e.g., monofractal box-counting methods or standard Detrended Fluctuation Analysis (DFA)). As brain disorders in humans are thought to reflect disorders of neuronal dynamics, multifractal DFA spectrum analysis of human EEG signals may also be used for accurate distinguishing of disorders of neuronal dynamics.

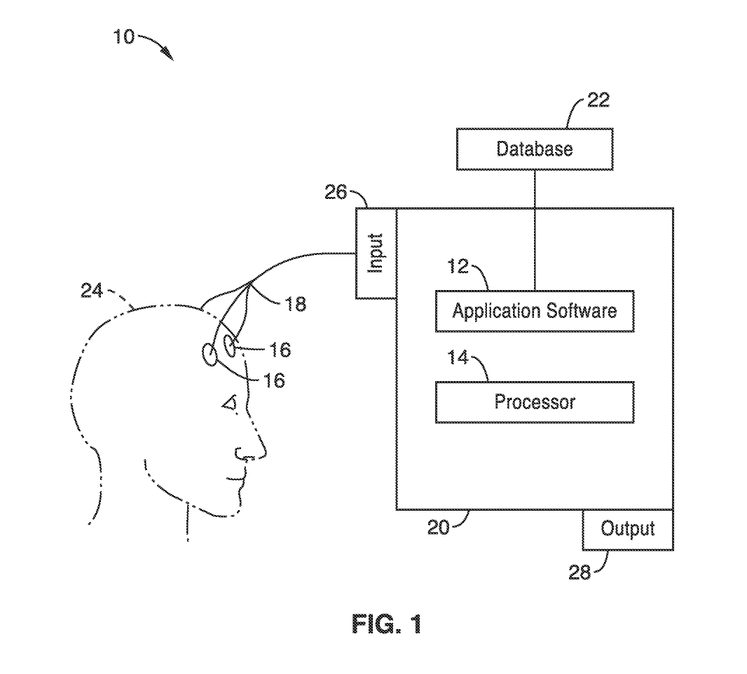

As shown in FIG. 1, the system and methods of the present invention may be implemented as system 10 configured to record an individual's EEG for a determined period of time, using standard EEG clinical practices. The EEG signals may be received through a plurality of leads 16 positioned on the patient's head 24. The leads are coupled to input 26 of processing apparatus 20 via lead wires 18. System 10 may be configured as an “EEG reader,” operating in much the same fashion as a commercially available electrocardiogram (EKG) machine.

System 10 would include a processing device 20 (e.g. computer or the like) comprising a specialized computer program/application 12 having one or more algorithms executable on processor 14 to perform the MF-DFA techniques on the recorded EEG signals. In a preferred embodiment, the application software 12 would further be configured to generate a multifractal DFA spectrum for each EEG signal. These spectra could then be compared to a database 22 of multifractal spectra of both normal individuals, and individuals with psychiatric and neurologic illness, to determine the likelihood that the test subject's EEG multifractal DFA spectra (derived from simultaneous multiple different scalp recordings) matches multifractal DFA spectra from the database derived from patients with (and without) known brain illnesses.

In a preferred embodiment, the application software 12 is configured to output 28 a “read” of the individual's EEG based upon the multifractal DFA spectra that would indicate the likelihood that the individual has a pattern consistent with either psychiatric or neurologic illness, in a manner similar to that currently available with EKG machines. The application software 12 may also be configured for monitoring stages of clinical anesthesia for surgical procedures, in that conscious awake states may be readily distinguishable from anesthetic states via multifractal DFA spectrum analysis.

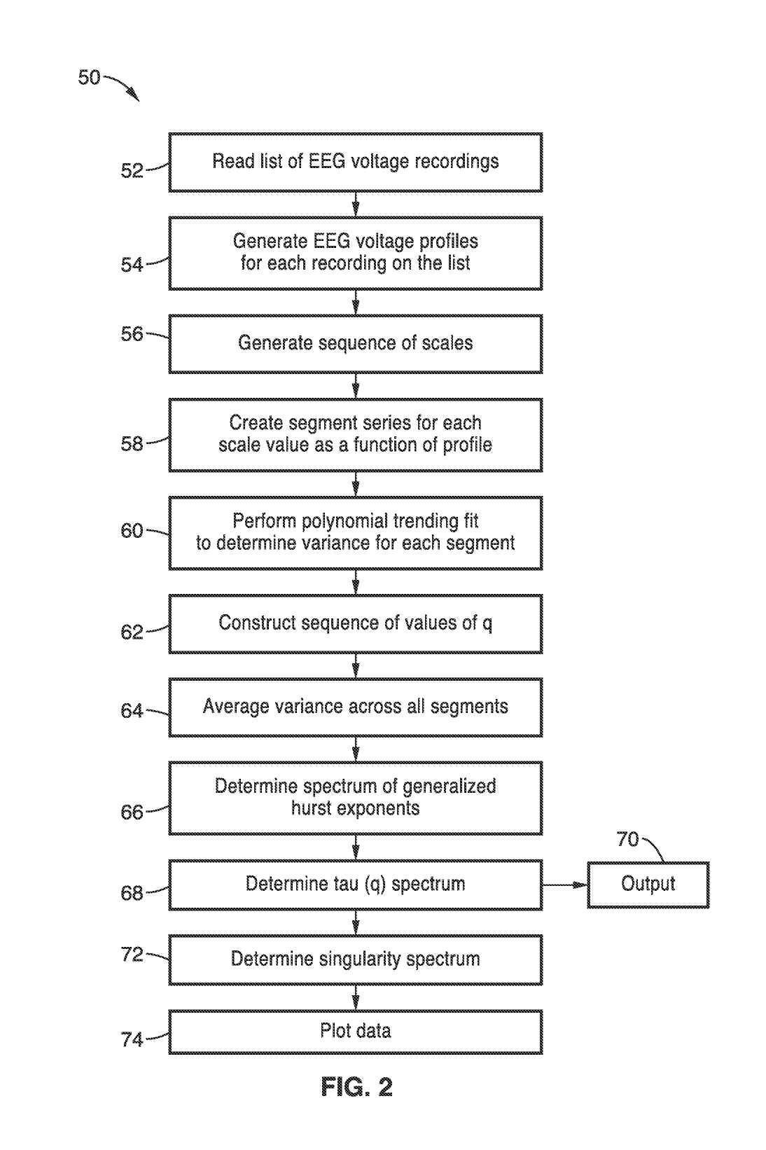

Referring now to FIG. 2, application software 12 may include an algorithm incorporating the method 50 for reading an individual's EEG based upon the multifractal DFA spectra. At step 52, a digitized list of sequential EEG voltage recordings are read as a function of time, wherein each reading is separated from the previous reading by a determined interval of time.

At step 54, the mean voltage of the entire list acquired in step 52 is calculated. This mean value is then subtracted from each individual voltage recording to compute the EEG “profile,” wherein the EEG profile is the sequence of the cumulative sums of mean-subtracted voltage recordings, each sum beginning with the first recording.

At step 56, the algorithm chooses a sequence of scales that will be used at a later time to determine the series trend as a function of scale. A scale is the length of a segment of consecutive data points. The scales range from several data points to roughly one fourth of the length of the list of voltage recordings.

At step 58, for each scale, the algorithm divides the profile into non-overlapping segments of equal scale, starting at the beginning of the profile. This operation is also performed in reverse order, starting from the end of the profile, such that there are two series of segments (one starting at the beginning, one starting at the end of the profile) for each scale.

At step 60, a separate fit is performed to the points within each segment of the profile mentioned in step 58 above, by a least-square fit to a polynomial of a given detrending order (e.g. linear, quadratic, cubic.). The fitted polynomial values from the profile is subtracted, and the variance of the residual values for each segment is determined (also referred as the detrending step). Detrending step 60 is repeated for each scale.

At step 62, a monotonic sequence of values q, ranging from maximum to minimum (usually a range smaller than −20 to 20) is then constructed.

At step 64, the variance to the q divided by the 2 power is calculated for each scale and each value q for every segment. This quantity is then averaged across all segments for each scale and each value q to generate the q^{th} order fluctuation function by taking this average value to the 1/q power.

At step 66, the spectrum of generalized Hurst exponents is determined by analyzing log-log plots of q^{th} order fluctuation functions versus scale. This is done for each value q in the sequence of q values. The slope of the linear fit of the log-log plot gives the “h” value or Hurst exponent for each value of q.

At step 68, tau(q) is calculated by multiplying the generalized Hurst exponent h by q for each value of q, and subtracting 1, i.e.:

tau(q)=q·h(q)−1 Eq. 1

In some embodiments, the plot of tau(q) versus q can be used as an alternative output function for the MF-DFA, and output at step 70.

At step 72, the singularity spectrum D(h) is determined from tau(q) via the Legendre transform, by taking the slope across all triplets of adjacent values for the graph of q vs. tau(q). The values of h or generalized Hurst exponents are also preferably rescaled to match the decreased length of the D(h) series as compared to the original spectrum of generalized Hurst exponents.

Finally, at step 74, the calculated data is output as a plot of one or more of q versus tau(q), q versus H(q), or h versus D(h). These plots provide a multifractal DFA spectrum that represents essential information regarding the long range correlations and fractal exponents that characterize the underlying cortical neuronal dynamics responsible for scalp-recorded EEG series.

Experiment #1: Sleep Studies

Studies were performed to investigate the use of MF-DFA as a tool to assess multifractality in human EEG, using sleep-stage data from a publicly available database. Several tests were performed to assess the efficacy of MF-DFA as a tool to characterize different sleep stages among subjects.

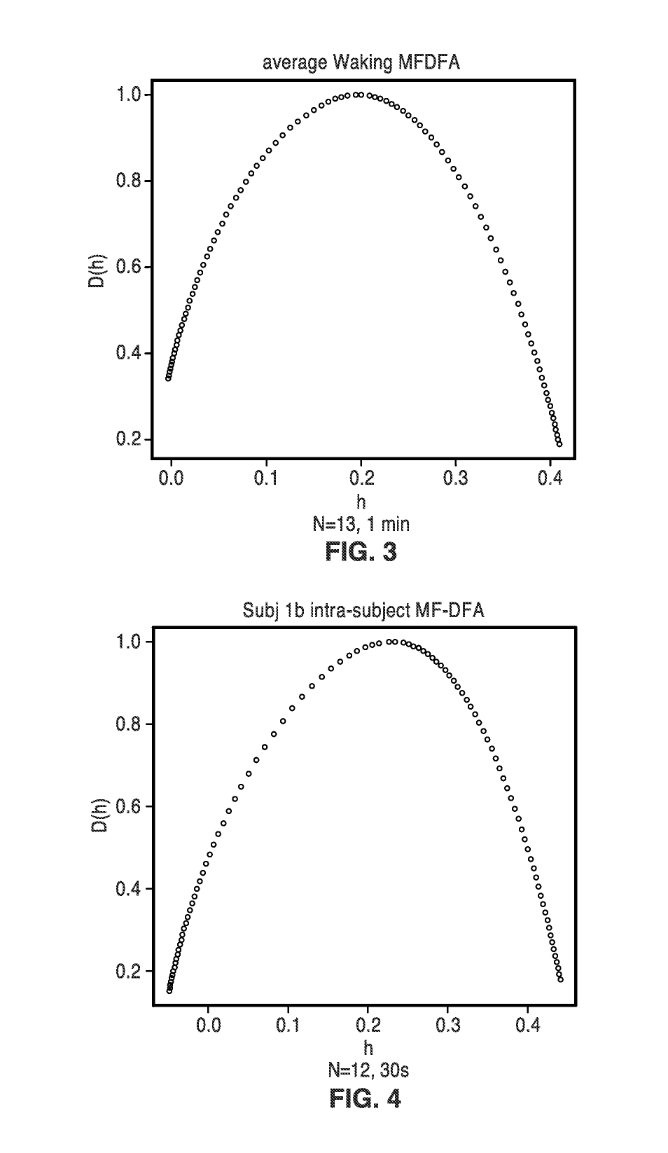

FIG. 3 shows a plot of the average multifractal DFA spectrum obtained from n=13 subjects, 1 minute of waking EEG per subject, showing the inverse parabola distribution of data for h versus D(h) plots.

FIG. 4 shows a plot of the average multifractal DFA spectrum obtained from 12 different 30 second segments of waking EEG from a single subject.

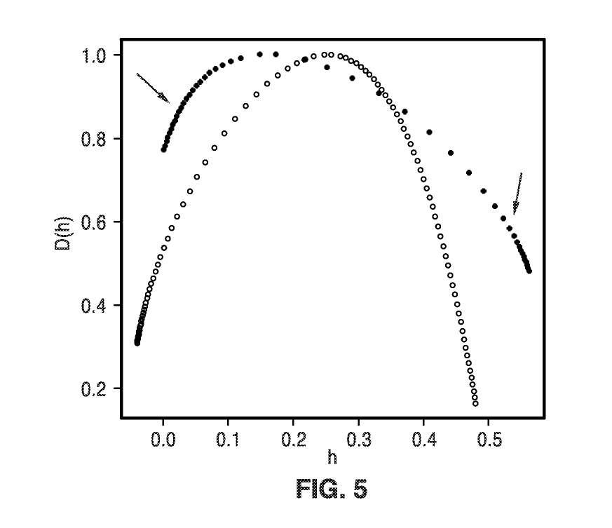

FIG. 5 is a plot of an exemplary multifractal DFA spectrum from a subject with waking EEG (∘) versus subject having a witnessed seizure (), generated from 15 seconds of EEG for each. The plot of h vs. D(h) is readily able to distinguish a patient having a seizure versus subject in normal waking state (arrows show regions of robust distinctiveness).

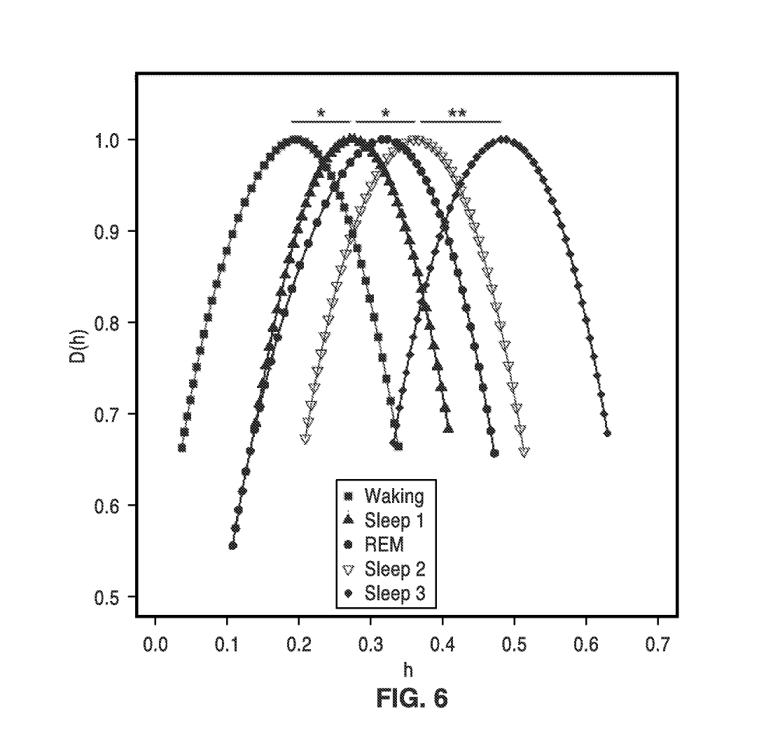

FIG. 6 is a plot of multifractal DFA spectra in different stages of sleep. Each graph represents averages of 1 minute of EEG data from subjects in various sleep stages: waking (n=12 subjects); sleep stage 1 (n=9 subjects); REM sleep (n=12 subjects); sleep stage 2 (n=15 subjects); and sleep stage 3 (n=13 subjects). Average MF-DFA spectra for each consciousness state shown here were calculated by averaging across individual spectrum values for each subject. Mean h values were then calculated for the h range, and differences between sleep stages compared by linear mixed effects modeling, with * corresponding to p<0.05 and ** corresponding to p<0.01. Significant differences were found between waking and sleep stage 1 EEGs (F_{(1,21.0)}=4.8, p=0.04), Sleep stage 1 and sleep stage 2 EEGs (F_{(1,8.6)}=8.4, p=0.019), and sleep stages 2 and sleep stage 3 EEGs (F_{(1,19.84)}=10.5, p=0.004).

Table 1 shows pairwise statistical comparisons for multifractal DFA h values between stages. Using only mean h values among the subjects from different stages, pairwise comparisons with bonferroni correction demonstrates significant group differences between h values for the different sleep stages.

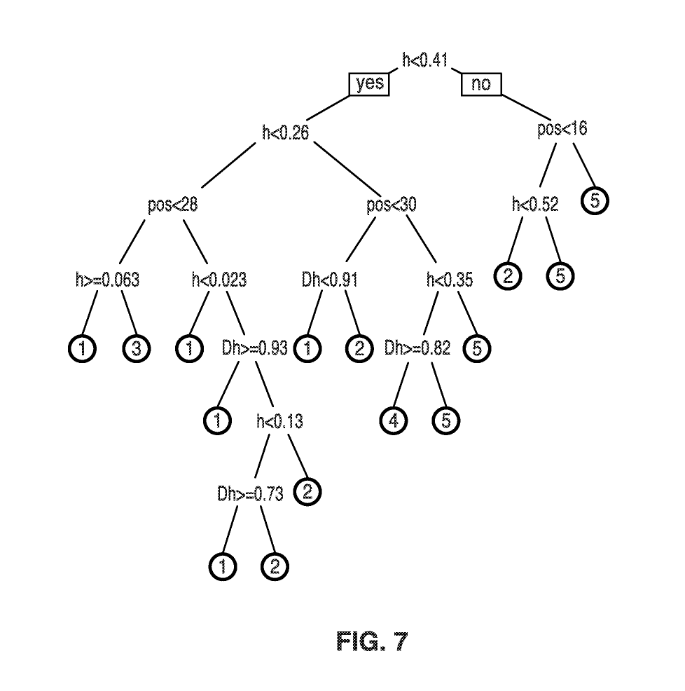

FIG. 7 shows a diagram of an exemplary classification tree algorithm for distinguishing sleep stages from multifractal DFA spectra of human EEG in accordance with the present invention. For each tree branch, the left hand side indicates those cases the listed branch condition is met (“yes”), the right hand side indicates those cases the listed branch condition is not met (“no”). Abbreviation (h) indicates h value; (pos) indicates the position on D(h) vs. h graph corresponding to the q value; (Dh) indicates D(h) value. Numbers in bubbles below tree branches indicate the likely classification of each sleep stage, given the classifications as follows: (1) sleep stage 1; (2) sleep stage 2; (3) sleep stage 3; (4) waking; (5) REM sleep.

Using a small training database of MF-DFA spectra from different sleep stages from 15 subjects, data from FIG. 6, and the technique of classification tree algorithm of FIG. 7, a simple heuristic algorithm was found that could correctly classify 100% of waking and sleep stage 3 EEG segments, and 92% of REM EEG segments, using as input data only multifractal DFA h values, D(h) values, and a marker for position. It should be noted that these results were achieved with very small training database, and it is expected that the accuracy should increase given larger datasets.

In summary, it was shown that even short EEG tracings of 30 s-1 m can have robust differences in multifractal spectra. Taken together, these data provide support for the possibility that analysis of EEG by MF-DFA may be a valuable tool in the automatic characterization of changes in brain and/or consciousness states.

Experiment #2: Comparison of MF-DFA with WTMM

Studies were conducted to compare multifractal detrended fluctuation analysis to a previously published multifractal technique, wavelet transform modulus maxima (WTMM), using EEG signals from waking and sleep.

Single channel EEG recordings with sleep stage annotations were downloaded from the MIT-BIH polysomnographic database (sampled at 256 Hz). The list of subject numbers and data utilized is provided in Table 1. Tracings were selected randomly based only upon relative lack of obvious movement artifacts. Both contiguous and non-contiguous tracings were joined together in 1 m (n=15000) segments that were annotated to be in the same consciousness state. Of the 16 possible subject records, only 14 had usable waking EEG tracings of >1 m in length (see Table 2).

Code for MF-DFA was written in the R programming language [R Core 39] following the method 50 shown in FIG. 2. While various ranges of q were tested, multifractal spectra were most consistent within the range of −5≦q≦5 (data not shown). Similarly, while higher-order polynomial detrending produced equivalent results, overall the spectra were well-characterized with a linear detrending procedure, which was thus exclusively utilized for this study (MF-DFA1; data not shown).

Code was also written for WTMM. To complement the MF-DFA analysis (see above), −5≦q≦5 was also used to generate multifractal spectra, with intervals of 0.2 units of q, such that multifractal spectra from both techniques were of the same length.

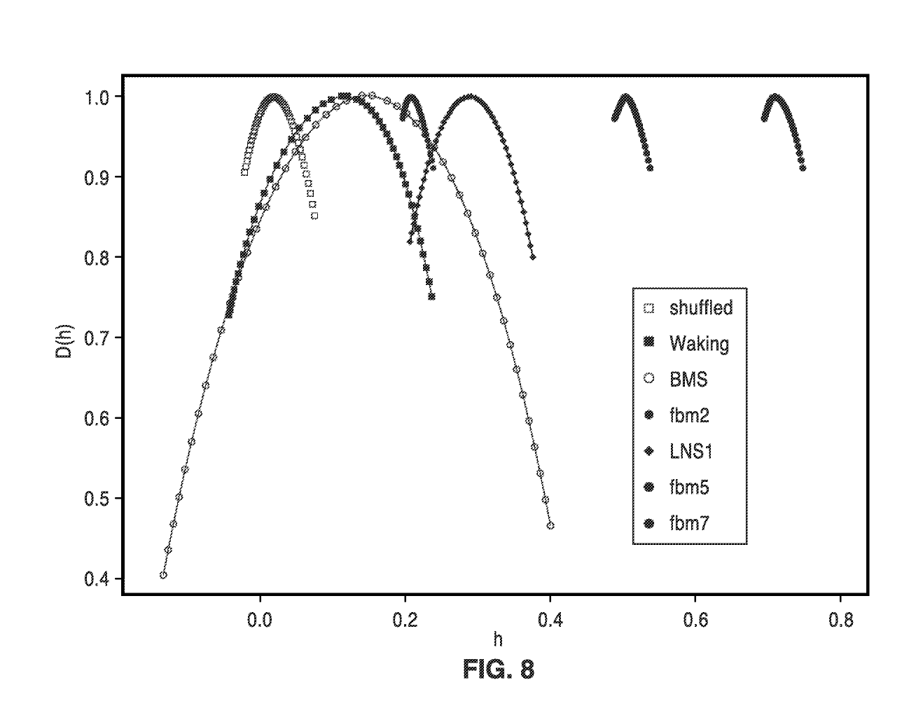

In the following, the h vs. D(h) naming convention is used, where h is the Holder exponent (abscissa) of a fractal subset and D(h) (ordinate) is the corresponding fractal dimension. For each time series, both analyses (MF-DFA and WTMM) produce spectra such as those shown in FIG. 8, each consisting of a set of 48 discrete points (h, D(h)) with inverted parabolic shape. For each spectrum the parameters mean_h and mean_D(h) were computed by averaging the points. Parapeter width_h was computed as the difference between the maximum h and the minimum h, and height_D(h) was computed as the difference between the maximum D(h) and minimum D(h).

Fractional Brownian motion monofractal series were generated with Hurst exponent (H) values of 0.2, 0.5 and 0.7 using the dvfBm R package (120,000 data points each; version 1.0). The binomial multifractal series was used, where a series of N=2ⁿ^{ₘₐₓ} numbers with index k=1, . . . , N, is defined by:

xₖ=a^{n(k-1)}(1−a)ⁿ^{ₘₐₓ}^{-n(k-1)}. Eq. 2

The parameter a=0.6 was chosen to roughly match the MF-DFA spectra from the EEG samples. Here n(k) is the sum of digits equal to 1 in the binary representation of the index k (120,000 data points, therefore nₘₐₓ=16.87). The log normal sigma 0.1 multifractal series (32,768 data points) made from the log-normal wavelet cascade algorithm with parameters v=ln(2)/4 and σ=0.1.

FIG. 8 is a plot showing a comparison of MF-DFA spectrum from waking EEG to numerical models of mono- and multifractal processes. Data points represent individual D(h) and h values from MF-DFA from a single time series of each type: (waking) waking EEG (8 m, n=120,000) from a single subject; (shuffled) waking EEG with values shuffled prior to MF-DFA analysis; (BMS) binomial multifractal series model with a=0.6 (n=120,000); (LNS1) log normal sigma 0.1 multifractal model data (n=32,768; (fbm2, 5, 7) fractional Brownian motion monofractal models with H_{b} values of 0.2, 0.5, 0.7 as indicated (n=120,000 each).

To assess the feasibility of using MF-DFA analysis on human EEG tracings, MF-DFA was performed on time series derived from 8 m long EEG tracings from subjects in the MIT-BIH slpdb database annotated for the waking state of consciousness (typical example from one subject presented in FIG. 8). For each time series, this analysis produced an MF-DFA spectrum of typical inverted parabolic shape with width_h invariably 0.21 units (FIG. 8; Table 3). Shuffling of the EEG time series followed by MF-DFA abolishes the multifractality (FIG. 8), resulting in a monofractal spectrum with mean_h of 0. In order to compare spectra derived from EEG with spectra derived from well-understood monofractal (fractional Brownian motion (fBm)) and multifractal series, the MF-DFA analysis was also performed on various fractal simulations. In all cases, the MF-DFA of fBm generated a narrow MF-DFA spectrum (<0.1 units), consistent with monofractality. By contrast, MF-DFA of both the binomial multifractal series and the log normal sigma multifractal series generated wider spectra (larger width_h) with a larger range of D(h) (larger height_D(h)) than the monofractal series (FIG. 8). By direct comparison, MF-DFA spectra of human waking EEG appear to have a degree of multifractality in between the two multifractal simulations, and clearly greater than those for the monofractal simulations (FIG. 8).

Table 3 shows the parameters derived from all 14 subjects' MF-DFA analyses on 8 m long waking EEG tracings.

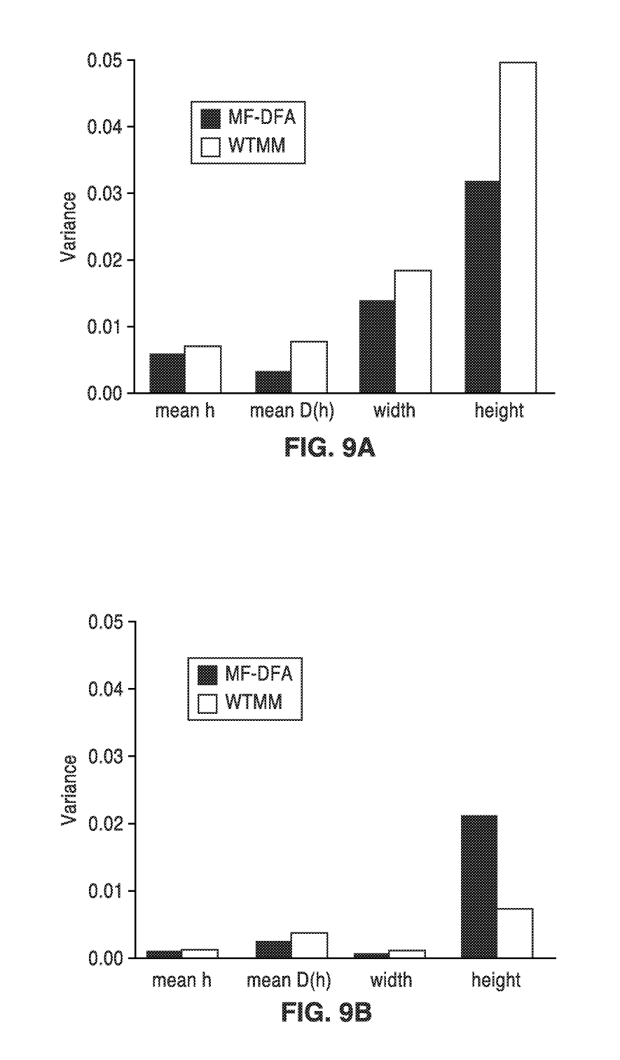

To directly compare the variability of multifractal spectral results from MF-DFA to that for WTMM, a MIT-BIH slpdb dataset comprised of 16 segments of 30 s each (7500 datapoints) of waking EEG derived from 14 subjects was used. FIG. 9A shows graphs of both types of multifractal analyses on each segment. For each multifractal spectrum from each segment, we calculated mean_h, mean_D(h), width_h, and height_D(h). The variances for MF-DFA were markedly decreased compared to those for WTMM. Estimates of the pooled estimated standard deviation were calculated for sample variances for each measure, and compared to the difference in sample variance between techniques as a ratio. Using a cutoff of >2 standard deviations more than the estimated standard deviation of the pooled estimate variances as a rough threshold for whether the measured difference in sample variances was likely to be meaningful, values of 1.3 for mean_h, 2.9 for width_h, 7.6 for mean_D(h), and 4.2 for height_D(h) were found. This indicates that the variances for the latter three measures were likely to be less for the MF-DFA technique than for the WTMM technique with 30 s EEG segments.

Referring now to FIG. 9B, this analysis was repeated with the entire 8 m EEG from each of 14 subjects, by comparing the variances derived from mean_h, width_h, mean_D(h), and height_D(h) for the MF-DFA and WTMM techniques. As expected, there was a strong trend for decreased variances overall for the longer tracings. Via the same estimation of the estimated standard deviation of the pooled estimated variances, compared to the measured difference in sample variances, values of 0.6 were found for mean_h, 0.4 for width_h, and 2.0 for mean_D(h), indicating that of these three measures, only the variance in mean_D(h) was likely to be lower for MF-DFA than for WTMM. By contrast, for the height_D(h) measure, a ratio of 3.3 was, indicating that for the 8 m tracing, the WTMM variance was likely to be lower than that for MF-DFA.

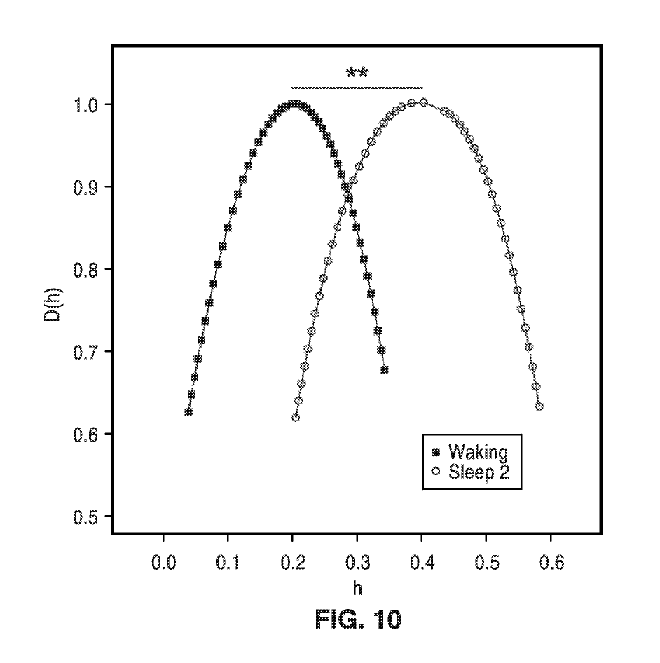

FIG. 10 is a plot comparing MF-DFA spectra of waking and sleep stage 2. For 14 subjects with 8 m of EEG from both waking and sleep stage 2 per subject, EEG was divided into 16 segments of 30 s each, and MF-DFA spectra were calculated for each segment (224 segments for each state of consciousness). Average MF-DFA spectra for each consciousness state shown here were calculated by averaging across individual spectrum values for each subject. **: p<0.001 for effect of state of consciousness by linear mixed effects modeling. MF-DFA spectra were also computed for each segment, and linear mixed effect modeling was used to perform comparisons both between states of consciousness, using data for mean_h, mean_D(h), width_h, and height_D(h) separately. For mean_h, there was a large difference between states of consciousness, with waking having smaller mean_h values (F_{(1,445)}=671, p<0.001). By contrast, there were no differences between sleep stages on width_h, mean_D(h) and height_D(h).

The results above suggest that MF-DFA may be more consistent than WTMM in terms of having a lower variance for parameters determined from multifractal spectral data for shorter recordings (30 s, or 7500 data points at 256 Hz, FIG. 9A), but being roughly consistent with WTMM for longer (8 m) recordings (FIG. 9B). Therefore, MF-DFA may be superior to WTMM in detecting changes in neuronal dynamics underlying changes of consciousness or perception via EEG in shorter recordings of ˜30 s.

MF-DFA may have utility in the recognition of changes in states of consciousness. The test results above support that MF-DFA analysis of even relatively short (˜1 m) EEG tracings may have sufficient sensitivity to assist in automatic recognition of changes in the state of consciousness, including sleep stages in polysomnography. Comparing differences in mean_h values is likely to be the most useful technique, given that these tend to vary more between different states of consciousness than mean_D(h) and other values.

The tests above suggest that multifractal analysis via MF-DFA of EEG signals recorded from humans may be used to gain an improved understanding of the relevant underlying neuronal dynamics, compared to traditional techniques. Given that cortical neuronal networks exhibit nonlinear interactions characterized by a range of fractal exponents with varying scales, the MF-DFA techniques of the present invention have the potential to distinguish essential features of the underlying neuronal dynamics for EEG signals in a way that is superior either to traditional techniques (e.g., spectral analysis via Fourier transform), or measures derived from monofractal analysis (e.g., monofractal box-counting methods or standard Detrended Fluctuation Analysis (DFA)). Brain disorders in humans are thought to reflect disorders of neuronal dynamics, and therefore multifractal DFA spectrum analysis of human EEG signals may prove to yield additional insights into disorders of neuronal dynamics than other currently available methods.

Experiment #3: Neurological Disorders

Tests were also conducted to determine the utility of the MF-DFA techniques of the present invention in identifying neurological disorders, and in particular, applications such as the diagnosis of the psychiatric disorder of Schizophrenia, the diagnoses of the neurological disorders of delirium, mild cognitive impairment (MCI) and dementia, and traumatic brain injury (TBI). It should be noted that in one each of the EEG tracings for one of the MCI/dementia patients and TBI patients that the official EEG reading by the neurologist was “normal EEG”, whereas this technique has shown a mean_h value outside of the range seen in healthy control subjects. Therefore, in addition to the information presented below, the MF-DFA techniques of the present invention may be superior to standard currently available techniques in the diagnosis of brain abnormalities associated with clinical diagnoses.

FIG. 11 illustrates a plot of MF-DFA spectra used in evaluation of schizophrenia. Schizophrenia diagnosis is characterized by a significantly higher h_max value than healthy control subjects in right parietal region. Eighteen healthy control (hc) subjects and 27 subjects with schizophrenia (scz) underwent two separate three-minute long EEG tracings. The first set was used to assess for the most explanatory differences in MF-DFA spectra among subjects utilizing the method of classification and regression trees (CART). These possible differences in individual leads were then analyzed by mixed linear model analysis in the second EEG set. This demonstrates that in the right parietal area, the maximum h value is significantly greater for subjects with schizophrenia than for healthy control subjects (p=0.013).

Note that this analysis represents a particular advantage of the multifractal analysis for EEG, in that standard monofractal analyses (representing a mean h value) would miss such a finding, as the mean h value does not differ between subject groups.

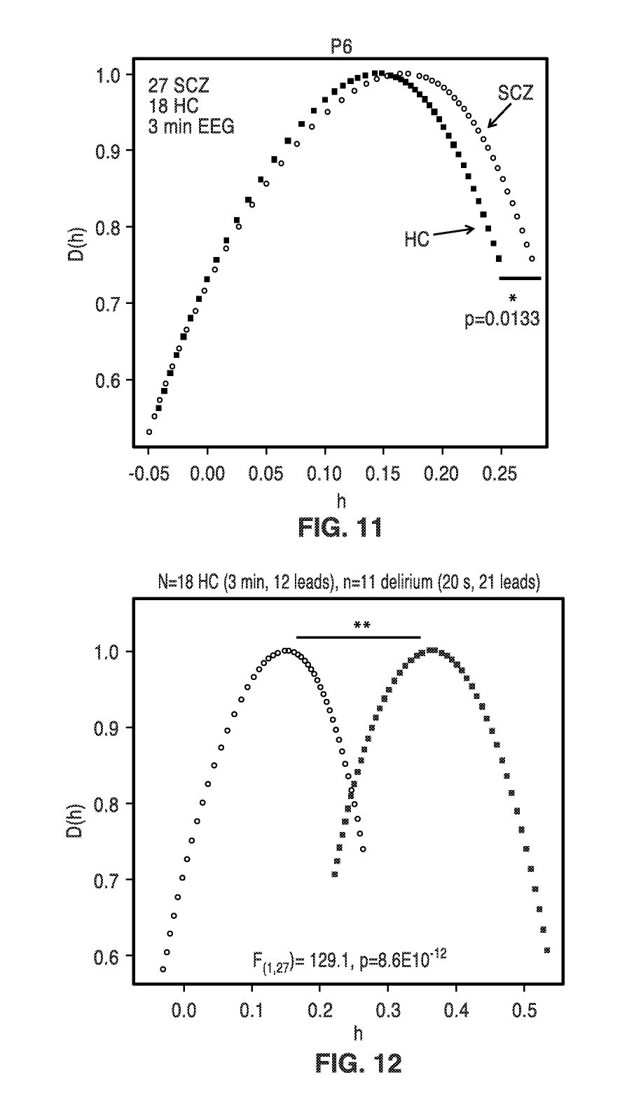

FIG. 12 illustrates a plot of MF-DFA spectra used in evaluation of delirium. Delirium diagnosis is characterized by a much larger mean_h value than healthy control subjects across leads. Average MFDFA spectra from 18 healthy control (hc) subjects and 11 subjects with delirium are plotted. HC subjects had 3 min of resting EEG (12 leads each), while delirium subjects had 20 sec of resting EEG (21 leads each). The data were compared using repeated measures ANOVA. This demonstrates that the mean_h value in delirium is much larger than in HC (p˜0).

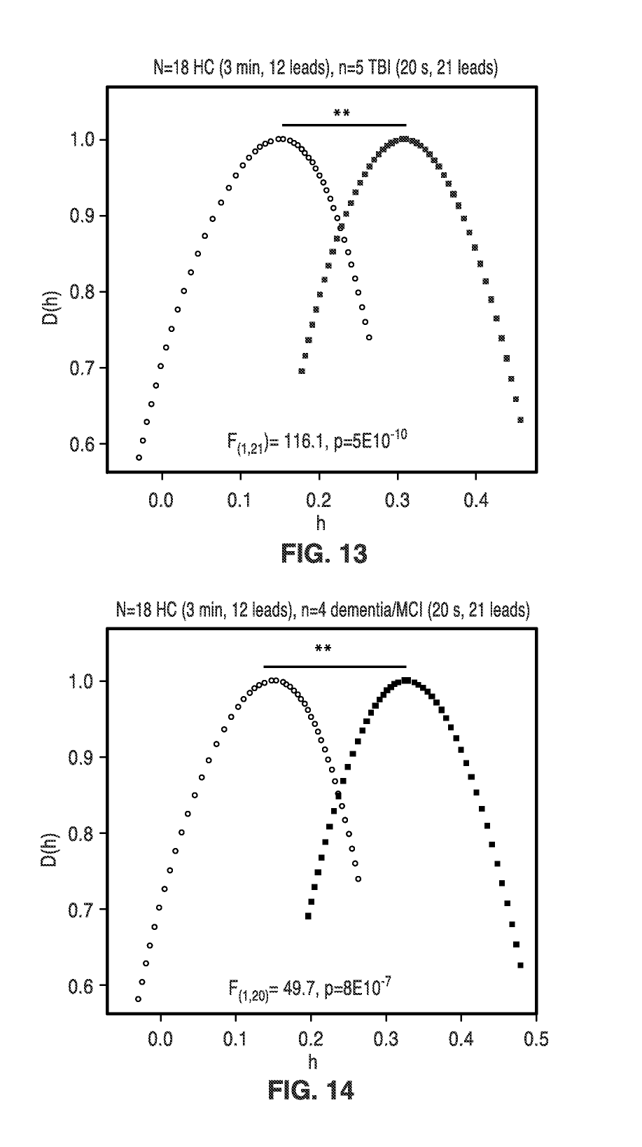

FIG. 13 illustrates a plot of MF-DFA spectra used in evaluation of Traumatic Brain Injury (TBI). History of Traumatic Brain Injury (TBI) is characterized by a much larger mean_h value than healthy control subjects across leads. Average MFDFA spectra from 18 healthy control (hc) subjects (black) and 5 subjects with TBI (green) are plotted. HC subjects had 3 min of resting EEG (12 leads each), while TBI subjects had 20 sec of resting EEG (21 leads each). The data were compared using repeated measures ANOVA. This demonstrates that the mean_h value in TBI is larger than in HC (p<10⁻⁹).

FIG. 14 illustrates a plot of MF-DFA spectra used in evaluation of Dementia and Mild Cognitive Impairment (MCI). Diagnosis of Mild Cognitive Impairment (MCI) and Dementia is characterized by a larger mean_h value than healthy control subjects across leads. Average MFDFA spectra from 18 healthy control (hc) subjects and 4 subjects with either MCI or dementia are plotted. HC subjects had 3 min of resting EEG (12 leads each), while MCI/dementia subjects had 20 sec of resting EEG (21 leads each). The data were compared using repeated measures ANOVA. This demonstrates that the mean_h value in MCI/dementia is larger than in HC (p<10-6).

Embodiments of the present invention may be described with reference to flowchart illustrations of methods and systems according to embodiments of the invention, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

It will further be appreciated that “programming” as used herein refers to one or more instructions that can be executed by a processor to perform a function as described herein. The programming can be embodied in software, in firmware, or in a combination of software and firmware. The programming can be stored local to the device in non-transitory media, or can be stored remotely such as on a server, or all or a portion of the programming can be stored locally and remotely. Programming stored remotely can be downloaded (pushed) to the device by user initiation, or automatically based on one or more factors, such as, for example, location, a timing event, detection of an object, detection of a facial expression, detection of location, detection of a change in location, or other factors. It will further be appreciated that as used herein, that the terms processor, central processing unit (CPU), and computer are used synonymously to denote a device capable of executing the programming and communication with input/output interfaces and/or peripheral devices.

From the discussion above it will be appreciated that the invention can be embodied in various ways, including but not limited to the following:

1. An apparatus for analyzing human electroencephalogram (EEG) signals, comprising: (a) a processor; and (b) programming executable on the processor and configured for: (i) acquiring a digitized set of sequential EEG voltage recordings as a function of time; (ii) performing multifractal-detrended fluctuation analysis (MF-DFA) on the set of sequential EEG voltage recordings; and (iii) outputting a MF-DFA spectrum corresponding to the set of sequential EEG voltage recordings.

2. An apparatus as in any of the previous embodiments, the programming further configured for: comparing the output MF-DFA spectrum against a database of MF-DFA spectrum to classify a neuronal state corresponding to the acquired set of sequential EEG voltage recordings.

3. An apparatus as in any of the previous embodiments, wherein the neuronal state comprises a sleep state of a patient.

4. An apparatus as in any of the previous embodiments, wherein the neuronal state comprises a psychiatric or neurologic disorder of a patient.

5. An apparatus as in any of the previous embodiments, wherein performing multifractal-detrended fluctuation analysis (MF-DFA) comprises: subtracting a mean voltage value from each EEG voltage recording in the set of sequential EEG voltage recordings to generate an EEG profile; selecting a sequence of scales corresponding to a length of a segment of consecutive data points within the EEG profile; for each scale, dividing the EEG profile into non-overlapping segments of equal scale; performing a fit to points within each segment of the EEG profile to a polynomial of a detrending order to generate a variance of residual values for each segment; constructing a sequence of q values; generating a spectrum of generalized Hurst exponents h for each value q in the sequence of q values; and generating a tau(q) spectrum as a function of each of the generalized Hurst exponents h for each value q.

6. An apparatus as in any of the previous embodiments, wherein performing multifractal-detrended fluctuation analysis (MF-DFA) comprises generating a plot of tau(q) versus q.

7. An apparatus as in any of the previous embodiments, wherein performing multifractal-detrended fluctuation analysis (MF-DFA) comprises: generating a singularity spectrum D(h) by computing a slope across adjacent values for the plot of tau(q) versus q; and generating a plot of one or more of q versus tau(q), q versus H(q), or h versus D(h).

8. An apparatus as in any of the previous embodiments, wherein the EEG profile is the sequence of the cumulative sums of mean-subtracted voltage recordings, each sum beginning with a first recording of the sequential EEG voltage recordings.

9. An apparatus as in any of the previous embodiments, wherein dividing the EEG profile into non-overlapping segments is performed from a beginning of the EEG profile to an end of the EEG profile, and then in reverse order from the end of the EEG profile to the beginning of the EEG profile to generate two series of segments.

10. An apparatus as in any of the previous embodiments, wherein performing a fit to points within each segment of the EEG profile comprises performing a least-square fit such that fitted polynomial values from the profile are subtracted, and a variance of the residual values for each segment is determined.

11. An apparatus as in any of the previous embodiments, wherein the spectrum of generalized Hurst exponents is determined by analyzing log-log plots of q^{th} order fluctuation functions versus scale for each value q in the sequence of q values.

12. An apparatus as in any of the previous embodiments, wherein a slope of a linear fit of the log-log plots gives an “h” value or Hurst exponent for each value of q.

13. An apparatus as in any of the previous embodiments, wherein tau(q) is calculated by multiplying a generalized Hurst exponent h by q for each value of q, and subtracting 1.

14. An apparatus as in any of the previous embodiments, wherein the singularity spectrum D(h) is determined from tau(q) via a Legendre transform as a function of the slope across all triplets of adjacent values for the graph of q vs. tau(q).

15. An apparatus for analyzing human EEG signals, comprising: (a) a processor; (b) programming executable on the processor and configured for: (i) acquiring a digitized set of sequential EEG voltage recordings as a function of time; (ii) subtracting a mean voltage value from each EEG voltage recording in the set of sequential EEG voltage recordings to generate an EEG profile; (iii) selecting a sequence of scales corresponding to a length of a segment of consecutive data points within the EEG profile; (iv) for each scale, dividing the EEG profile into non-overlapping segments of equal scale; (v) performing a fit to points within each segment of the EEG profile to a polynomial of a detrending order to generate a variance of residual values for each segment; (vi) constructing a sequence of q values; (vii) generating a spectrum of generalized Hurst exponents h for each value q in the sequence of q values; and (viii) generating a MF-DFA tau(q) spectrum as a function of each of the generalized Hurst exponents h for each value q.

16. An apparatus as in any of the previous embodiments, the programming further configured for: comparing the output MF-DFA spectrum against a database of MF-DFA spectrum to classify a neuronal state corresponding to the acquired set of sequential EEG voltage recordings.

17. An apparatus as in any of the previous embodiments, wherein the neuronal state comprises a sleep state of a patient.

18. An apparatus as in any of the previous embodiments, wherein the neuronal state comprises a psychiatric or neurologic disorder of a patient.

19. An apparatus as in any of the previous embodiments, wherein the MF-DFA spectrum comprises a tau(q) spectrum calculated from a spectrum of generalized Hurst exponents determined by analyzing log-log plots of q^{th} order fluctuation functions versus scale for each value q in the sequence of q values.

20. An apparatus as in any of the previous embodiments, the programming further configured for: generating a singularity spectrum D(h) by computing a slope across adjacent values for the plot of tau(q) versus q; and generating a plot of one or more of q versus tau(q), q versus H(q), or h versus D(h).

21. An apparatus as in any of the previous embodiments, wherein the EEG profile is the sequence of the cumulative sums of mean-subtracted voltage recordings, each sum beginning with a first recording of the sequential EEG voltage recordings.

22. An apparatus as in any of the previous embodiments, wherein dividing the EEG profile into non-overlapping segments is performed from a beginning of the EEG profile to an end of the EEG profile, and then in reverse order from the end of the EEG profile to the beginning of the EEG profile to generate two series of segments.

23. An apparatus as in any of the previous embodiments, wherein performing a fit to points within each segment of the EEG profile comprises performing a least-square fit such that fitted polynomial values from the profile are subtracted, and a variance of the residual values for each segment is determined.

24. An apparatus as in any of the previous embodiments, wherein a slope of a linear fit of the log-log plots gives an “h” value or Hurst exponent for each value of q.

25. An apparatus as in any of the previous embodiments, wherein tau(q) is calculated by multiplying a generalized Hurst exponent h by q for each value of q, and subtracting 1.

26. An apparatus as in any of the previous embodiments, wherein the singularity spectrum D(h) is determined from tau(q) via a Legendre transform as a function of the slope across all triplets of adjacent values for the graph of q vs. tau(q).

27. A method for analyzing human EEG signals, comprising: acquiring a digitized set of sequential EEG voltage recordings as a function of time; subtracting a mean voltage value from each EEG voltage recording in the set of sequential EEG voltage recordings to generate an EEG profile; selecting a sequence of scales corresponding to a length of a segment of consecutive data points within the EEG profile; for each scale, dividing the EEG profile into non-overlapping segments of equal scale; performing a fit to points within each segment of the EEG profile to a polynomial of a detrending order to generate a variance of residual values for each segment; constructing a sequence of q values; generating a spectrum of generalized Hurst exponents h for each value q in the sequence of q values; and generating a MF-DFA tau(q) spectrum as a function of each of the generalized Hurst exponents h for each value q.

28. A method as in any of the previous embodiments, further comprising: comparing the output MF-DFA spectrum against a database of MF-DFA spectrum to classify a neuronal state corresponding to the acquired set of sequential EEG voltage recordings.

29. A method as in any of the previous embodiments, wherein the neuronal state comprises a sleep state of a patient.

30. A method as in any of the previous embodiments, wherein the neuronal state comprises a psychiatric or neurologic disorder of a patient.

31. A method as in any of the previous embodiments, wherein the MF-DFA spectrum comprises a tau(q) spectrum calculated from a spectrum of generalized Hurst exponents determined by analyzing log-log plots of q^{th} order fluctuation functions versus scale for each value q in the sequence of q values.

32. A method as in any of the previous embodiments, the programming further configured for: generating a singularity spectrum D(h) by computing a slope across adjacent values for the plot of tau(q) versus q; and generating a plot of one or more of q versus tau(q), q versus H(q), or h versus D(h).

33. A method as in any of the previous embodiments, wherein the EEG profile is the sequence of the cumulative sums of mean-subtracted voltage recordings, each sum beginning with a first recording of the sequential EEG voltage recordings.

34. A method as in any of the previous embodiments, wherein dividing the EEG profile into non-overlapping segments is performed from a beginning of the EEG profile to an end of the EEG profile, and then in reverse order from the end of the EEG profile to the beginning of the EEG profile to generate two series of segments.

35. A method as in any of the previous embodiments, wherein performing a fit to points within each segment of the EEG profile comprises performing a least-square fit such that fitted polynomial values from the profile are subtracted, and a variance of the residual values for each segment is determined.

36. A method as in any of the previous embodiments, wherein a slope of a linear fit of the log-log plots gives an “h” value or Hurst exponent for each value of q.

37. A method as in any of the previous embodiments, wherein tau(q) is calculated by multiplying a generalized Hurst exponent h by q for each value of q, and subtracting 1.

38. A method as in any of the previous embodiments, wherein the singularity spectrum D(h) is determined from tau(q) via a Legendre transform as a function of the slope across all triplets of adjacent values for the graph of q vs. tau(q).

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for.” No claim element herein is to be construed as a “step plus function” element unless the element is expressly recited using the phrase “step for.”

TABLE 1

| | Wake | REM | Sleep1 | Sleep2 |
|---|---|---|---|---|
| REM | <0.001 | | | |
| Sleep1 | <0.001 | 0.023 | | |
| Sleep2 | <0.001 | <0.001 | <0.001 | |
| Sleep3 | <0.001 | <0.001 | <0.001 | <0.001 |

TABLE 2

| | 8 min | | | | | | |
|---|---|---|---|---|---|---|---|
| | waking/ | | 1 min sleep stage data: | | | | |
| | sleep 2 | recording | | Sleep | | | |
| Subject | data | site | waking | 1 | sleep 2 | sleep 3 | REM |
| S1 | yes | C4-A1 | yes | yes | yes | no | yes |
| S2 | yes | O2-A1 | yes | no | yes | no | yes |
| S3 | yes | C3-O1 | yes | yes | yes | no | yes |
| S4 | yes | C3-O1 | yes | yes | yes | yes | yes |
| S14 | yes | C3-O1 | yes | yes | yes | yes | yes |
| S16 | yes | C3-O1 | yes | no | yes | yes | yes |
| S32 | yes | C4-A1 | yes | yes | yes | yes | no |
| S37 | yes | C4-A1 | yes | no | yes | no | no |
| S41 | yes | C4-A1 | yes | yes | yes | yes | yes |
| S45 | no | C3-O1 | no | no | yes | yes | yes |
| S48 | yes | C3-O1 | yes | yes | yes | no | yes |
| S59 | yes | C3-O1 | yes | no | yes | yes | yes |
| S60 | yes | C3-O1 | yes | yes | yes | no | yes |
| S61 | yes | C3-O1 | yes | yes | yes | yes | yes |
| S66 | yes | C3-O1 | yes | no | yes | no | no |

TABLE 3

| Subject | mean_h | width_h | mean_D(h) | height_D(h) |
|---|---|---|---|---|
| S1 | 0.099 | 0.28 | 0.884 | 0.273 |
| S2 | 0.105 | 0.232 | 0.902 | 0.403 |
| S3 | 0.203 | 0.332 | 0.848 | 0.642 |
| S4 | 0.078 | 0.254 | 0.888 | 0.471 |
| S14 | 0.147 | 0.278 | 0.891 | 0.272 |
| S16 | 0.128 | 0.259 | 0.900 | 0.231 |
| S32 | 0.112 | 0.379 | 0.832 | 0.539 |
| S37 | 0.101 | 0.271 | 0.885 | 0.522 |
| S41 | 0.093 | 0.245 | 0.890 | 0.418 |
| S48 | 0.106 | 0.324 | 0.856 | 0.497 |
| S59 | 0.129 | 0.334 | 0.855 | 0.556 |
| S60 | 0.099 | 0.249 | 0.892 | 0.335 |
| S61 | 0.14 | 0.211 | 0.904 | 0.735 |
| S66 | 0.136 | 0.239 | 0.890 | 0.498 |
| mean (s.d.) | 0.12 (0.03) | 0.278 (0.05) | 0.880 (0.02) | 0.456 (0.140) |

What is claimed is:

1. An apparatus for analyzing human electroencephalogram (EEG) signals, comprising:
(a) a processor; and
(b) programming executable on the processor and configured for:
(i) acquiring a digitized set of sequential EEG voltage recordings as a function of time;
(ii) performing multifractal-detrended fluctuation analysis (MF-DFA) on the set of sequential EEG voltage recordings; and
(iii) outputting a MF-DFA spectrum corresponding to the set of sequential EEG voltage recordings.

2. An apparatus as recited in claim 1, the programming further configured for comparing the output MF-DFA spectrum against a database of MF-DFA spectrum to classify a neuronal state corresponding to the acquired set of sequential EEG voltage recordings.

3. An apparatus as recited in claim 2, wherein the neuronal state comprises a sleep state of a patient.

4. An apparatus as recited in claim 2, wherein the neuronal state comprises a psychiatric or neurologic disorder of a patient.

5. An apparatus as recited in claim 1, wherein performing multifractal-detrended fluctuation analysis (MF-DFA) comprises:
subtracting a mean voltage value from each EEG voltage recording in the set of sequential EEG voltage recordings to generate an EEG profile;
selecting a sequence of scales corresponding to a length of a segment of consecutive data points within the EEG profile;
for each scale, dividing the EEG profile into non-overlapping segments of equal scale;
performing a fit to points within each segment of the EEG profile to a polynomial of a detrending order to generate a variance of residual values for each segment;
constructing a sequence of q values;
generating a spectrum of generalized Hurst exponents h for each value q in the sequence of q values; and
generating a tau(q) spectrum as a function of each of the generalized Hurst exponents h for each value q.

6. An apparatus as recited in claim 5, wherein performing multifractal-detrended fluctuation analysis (MF-DFA) comprises generating a plot of tau(q) versus q.

7. An apparatus as recited in claim 6, wherein performing multifractal-detrended fluctuation analysis (MF-DFA) comprises:
generating a singularity spectrum D(h) by computing a slope across adjacent values for the plot of tau(q) versus q; and
generating a plot of one or more of q versus tau(q), q versus H(q), or h versus D(h).

8. An apparatus as recited in claim 5, wherein the EEG profile is the sequence of the cumulative sums of mean-subtracted voltage recordings, each sum beginning with a first recording of the sequential EEG voltage recordings.

9. An apparatus as recited in claim 5, wherein dividing the EEG profile into non-overlapping segments is performed from a beginning of the EEG profile to an end of the EEG profile, and then in reverse order from the end of the EEG profile to the beginning of the EEG profile to generate two series of segments.

10. An apparatus as recited in claim 5, wherein performing a fit to points within each segment of the EEG profile comprises performing a least-square fit such that fitted polynomial values from the profile are subtracted, and a variance of the residual values for each segment is determined.

11. An apparatus as recited in claim 5, wherein the spectrum of generalized Hurst exponents is determined by analyzing log-log plots of q^{th} order fluctuation functions versus scale for each value q in the sequence of q values.

12. An apparatus as recited in claim 11, wherein a slope of a linear fit of the log-log plots gives an “h” value or Hurst exponent for each value of q.

13. An apparatus as recited in claim 12, wherein tau(q) is calculated by multiplying a generalized Hurst exponent h by q for each value of q, and subtracting 1.

14. An apparatus as recited in claim 12, wherein the singularity spectrum D(h) is determined from tau(q) via a Legendre transform as a function of the slope across all triplets of adjacent values for the graph of q vs. tau(q).

15. An apparatus for analyzing human EEG signals, comprising:
(a) a processor;
(b) programming executable on the processor and configured for:
(i) acquiring a digitized set of sequential EEG voltage recordings as a function of time;
(ii) subtracting a mean voltage value from each EEG voltage recording in the set of sequential EEG voltage recordings to generate an EEG profile;
(iii) selecting a sequence of scales corresponding to a length of a segment of consecutive data points within the EEG profile;
(iv) for each scale, dividing the EEG profile into non-overlapping segments of equal scale;
(v) performing a fit to points within each segment of the EEG profile to a polynomial of a detrending order to generate a variance of residual values for each segment;
(vi) constructing a sequence of q values;
(vii) generating a spectrum of generalized Hurst exponents h for each value q in the sequence of q values; and
(viii) generating a MF-DFA tau(q) spectrum as a function of each of the generalized Hurst exponents h for each value q.

16. An apparatus as recited in claim 15, the programming further configured for comparing the output MF-DFA spectrum against a database of MF-DFA spectrum to classify a neuronal state corresponding to the acquired set of sequential EEG voltage recordings.

17. An apparatus as recited in claim 16, wherein the neuronal state comprises a sleep state of a patient.

18. An apparatus as recited in claim 16, wherein the neuronal state comprises a psychiatric or neurologic disorder of a patient.

19. An apparatus as recited in claim 15, wherein the MF-DFA spectrum comprises a tau(q) spectrum calculated from a spectrum of generalized Hurst exponents determined by analyzing log-log plots of q^{th} order fluctuation functions versus scale for each value q in the sequence of q values.

20. An apparatus as recited in claim 19, the programming further configured for:
generating a singularity spectrum D(h) by computing a slope across adjacent values for the plot of tau(q) versus q; and
generating a plot of one or more of q versus tau(q), q versus H(q), or h versus D(h).

21. An apparatus as recited in claim 15, wherein the EEG profile is the sequence of the cumulative sums of mean-subtracted voltage recordings, each sum beginning with a first recording of the sequential EEG voltage recordings.

22. An apparatus as recited in claim 15, wherein dividing the EEG profile into non-overlapping segments is performed from a beginning of the EEG profile to an end of the EEG profile, and then in reverse order from the end of the EEG profile to the beginning of the EEG profile to generate two series of segments.

23. An apparatus as recited in claim 15, wherein performing a fit to points within each segment of the EEG profile comprises performing a least-square fit such that fitted polynomial values from the profile are subtracted, and a variance of the residual values for each segment is determined.

24. An apparatus as recited in claim 19, wherein a slope of a linear fit of the log-log plots gives an “h” value or Hurst exponent for each value of q.

25. An apparatus as recited in claim 24, wherein tau(q) is calculated by multiplying a generalized Hurst exponent h by q for each value of q, and subtracting 1.

26. An apparatus as recited in claim 25, wherein the singularity spectrum D(h) is determined from tau(q) via a Legendre transform as a function of the slope across all triplets of adjacent values for the graph of q vs. tau(q).

27. A method for analyzing human EEG signals, comprising:
acquiring a digitized set of sequential EEG voltage recordings as a function of time;
subtracting a mean voltage value from each EEG voltage recording in the set of sequential EEG voltage recordings to generate an EEG profile;
selecting a sequence of scales corresponding to a length of a segment of consecutive data points within the EEG profile;
for each scale, dividing the EEG profile into non-overlapping segments of equal scale;
performing a fit to points within each segment of the EEG profile to a polynomial of a detrending order to generate a variance of residual values for each segment;
constructing a sequence of q values;
generating a spectrum of generalized Hurst exponents h for each value q in the sequence of q values; and
generating a MF-DFA tau(q) spectrum as a function of each of the generalized Hurst exponents h for each value q.

28. A method as recited in claim 27, further comprising:
comparing the output MF-DFA spectrum against a database of MF-DFA spectrum to classify a neuronal state corresponding to the acquired set of sequential EEG voltage recordings.

29. A method as recited in claim 28, wherein the neuronal state comprises a sleep state of a patient.

30. A method as recited in claim 28, wherein the neuronal state comprises a psychiatric or neurologic disorder of a patient.

31. A method as recited in claim 27, wherein the MF-DFA spectrum comprises a tau(q) spectrum calculated from a spectrum of generalized Hurst exponents determined by analyzing log-log plots of q^{th} order fluctuation functions versus scale for each value q in the sequence of q values.

32. A method as recited in claim 31, the programming further configured for:
generating a singularity spectrum D(h) by computing a slope across adjacent values for the plot of tau(q) versus q; and
generating a plot of one or more of q versus tau(q), q versus H(q), or h versus D(h).

33. A method as recited in claim 27, wherein the EEG profile is the sequence of the cumulative sums of mean-subtracted voltage recordings, each sum beginning with a first recording of the sequential EEG voltage recordings.

34. A method as recited in claim 27, wherein dividing the EEG profile into non-overlapping segments is performed from a beginning of the EEG profile to an end of the EEG profile, and then in reverse order from the end of the EEG profile to the beginning of the EEG profile to generate two series of segments.

35. A method as recited in claim 27, wherein performing a fit to points within each segment of the EEG profile comprises performing a least-square fit such that fitted polynomial values from the profile are subtracted, and a variance of the residual values for each segment is determined.

36. A method as recited in claim 31, wherein a slope of a linear fit of the log-log plots gives an “h” value or Hurst exponent for each value of q.

37. A method as recited in claim 36, wherein tau(q) is calculated by multiplying a generalized Hurst exponent h by q for each value of q, and subtracting 1.

38. A method as recited in claim 37, wherein the singularity spectrum D(h) is determined from tau(q) via a Legendre transform as a function of the slope across all triplets of adjacent values for the graph of q vs. tau(q).
